(19) United States
(12) Patent Application Publication
(10) Pub. No.: US 2004/0043496 A1
(43) Pub. Date: Mar. 4, 2004

(54) Method for identifying patients who will require multiple invasive cardiovascular procedures

(75) Inventor: H. Robert Superko, Burlingame, CA (US)

Correspondence Address: John J. McDonnell, McDonnell Boehnen Hulbert & Berghoff, 32nd Floor, 300 S. Wacker Drive, Chicago, IL, 60606, US

(73) Assignee: Berkeley HeartLab, Inc., Burlingame, CA

(21) Appl. No.: 10/463,664

(22) Filed: Jun. 16, 2003

Related U.S. Application Data
(60) Provisional application No. 60/390,796, filed on Jun. 21, 2002.

(51) Int. Cl. G01N 33/92

(52) U.S. Cl. 436/71

(57) ABSTRACT

The invention provides a method for identifying patients who will require multiple invasive cardiac procedures comprising measuring elevated LDL IVb levels in patients who have had or will have invasive heart surgery.

This application claims priority of U.S. Provisional Application 60/390,796 filed Jun. 21, 2002, and is incorporated herein by referenced in its entirety.

### FIELD OF THE INVENTION

This invention is in the field of cardiovascular healthcare management and patient treatment.

### BACKGROUND OF THE INVENTION

The invention utilizes the health care management system described in WO 01/41037A3 to study data from patient populations for cardiovascular risk factors especially those factors related to LDL and HDL subclass and in particular LDL-IVb. WO 01/41037AC is incorporated herein in its entirety. The text, Heart Disease Breakthrough, by Thomas Yannios, M.D. John Wiley & Son, Inc., New York, 1999 discusses management of heart disease and the role of HDL and LDL subclasses and is incorporated herein by reference.

Superko H R. Sophisticated tests for susceptibility to atherosclerosis: An added value for Heart Hospitals Am Heart Hospt J. 2003; 1:135-140.

Brown C, Garrett B, Superko H R. Small LDL and hs-CRP as New CAD Risk Factors. Journal of the Medical Association of Georgia 2001;90: 11-18.

Brown C, Garrett B, Superko H R. Small LDL and hs-CRP as New CAD Risk Factors. Journal of the Medical Association of Georgia 2001;90:11-18.

Superko H R. Hypercholesterolemia and Dyslipidemia. Current Treatment Options in Cardiovascular Medicine 2000;2:173-187.

Superko H R. Lipid Altering Drugs LDL/HDL subclass Distribution. Match the Treatment to the Disorder. ACC Current Journal Review 2000;9:3:18-24.

Williams, P T, Superko, H R, Alderman E A, Small Low Density Lipoprotein III but not Low Density Lipoprotein Cholesterol is Related to Arteriographic Progression, Circulation 2000; 102:II-848

Annual rate of stenosis change was related concordantly to on-study averages of total cholesterol (p=0.04), triglycerides (p=0.05), VLDL-mass (p=0.03), total HDLC ratio (p=0.04), LDL-IVb (p=0.01) and HDL3a (p=0.02) and inversely to HDL2mass (p=0.02) and HDL2b (p=0.03). Adjustment for other lipid and lipoprotein factors including most other lipoprotein subclasses, did not eliminate the significant association between LDL-IVb and the annual rate of stenosis change. Stepwise multiple regression analysis showed that the best fitting model for predicting stenosis change included only one lipoprotein variable; LDL-IVb. The average annual rate in stenosis change was six-fold more rapid in the fourth quartile of LDL-IVb (>5.2%) than in the first quartile (>2.5%, p=0.03). Analysis of patients with existing CAD reveals that those with LDL IVb>10% exhibit significantly more repeat clinical cardiovascular events (p<0.0001).

### SUMMARY OF THE INVENTION

In analyzing LDL and HDL subclass data from more than 80,000 cardiovascular patients, it has been found that indicia for patient treatment can be derived from LDL and HDL subclass information that is not available from NCEP risk factor data. Thus, the invention permits the identification of patients who have an undesirable small dense LDL trait and an impaired reverse cholesterol transport system. For example, it has unexpectedly been found for the first time that elevated LDL-IVb levels are predictive of the need for multiple invasive cardiac procedures. This analysis indicates that in patients requiring invasive cardiac procedures who have a high amount of LDL distribution in the IVb region are likely to need repeated invasive procedures despite lower LDL-C values. A metabolic profile characterized by an abundance of the smallest LDL particles appears to increase the risk of a patient for a need for repeated cardiovascular procedures.

Thus, the invention relates to identifying patients who will or who have had an invasive cardiovascular procedure who have a LDL IVb level greater than 10% of the LDL distribution.

LDL IVb subclass may be determined by linear gel electrophoresis, gradient gel electrophoresis (GGE), NMR, analytical ultracentrifugation, or ion mobility analysis.

The subfraction levels may be identified by any means such as per cent distribution of the subfraction levels or with quantitation such as concentration of a subfraction level or with the size of the predominant lipoprotein molecules. Once a patient has been identified (diagnosed), the method is used to generate an individualized treatment plan for the patient that may include drugs, diet and exercise.

### DETAILED DESCRIPTION OF THE INVENTION

The relationship of elevated LDL IVb in a group of patients who required invasive procedure(s) has been investigated. Methods: 79 (Cardiovascular Disease) CAD patients requiring cardiac intervention procedure, were randomly selected based on LDL IVb>=10% or =<3% of the total LDL distribution. Fasting Triglycerides, LDL-C and HDL-C were determined using enzymatic methods, LDL subclass distribution by S3-gradient gel electrophoresis, and Lp(a), homocysteine, and insulin by immunochemical methods. Results: There were no significant differences between groups in regard to age, total cholesterol, Lp(a), Apo B, and fibrinogen. The fasting insulin and hs-CRP values were higher (+) 149%, p+0.06) and (+221%, p+0.06) in the IVb+>10% group, (see Table).

| | IVb < = 3% | IVb ≧ 10% | | |
|---|---|---|---|---|
| N | 30 | 49 | % | P |
| # procedures | 0.2 ± 0.5 | 1.3 ± 1.6 | +650% | 0.0001 |
| LDL IVb (%) | 1.8 ± 0.8 | 12.7 ± 2.4 | +706% | 0.0001 |
| LDL IVa (%) | 3.5 ± 2.4 | 11.3 ± 4.4 | +323% | 0.0001 |
| Triglycerides | 116 ± 73 | 314 ± 245 | +271 | 0.0001 |
| (mg/dl) | | | | |
| HDL-C (mg/dl) | 50.0 ± 15.6 | 35.7 ± 11.6 | −40% | 0.0001 |
| LDL-C (mg/dl) | 98.0 ± 32 | 76.7 ± 22 | −28% | 0.002 |

Thus, patients who need or who have had an invasive cardiovascular procedure such as angioplasty are monitored for the level of LDL IVb. Levels above 10% of LDL IVb is predictive of patients who are more likely to need multiple invasive procedures such as angioplasty. Invasive procedures refer to angioplasty, by-pass and similar invasive procedures to treat cardiovascular disease.

What is claimed is:

1. A method for identifying patients who will need multiple invasive cardiovascular procedures comprising determining the elevated LDL IVb levels.

2. The method of claim 1 wherein the elevated level of LDL IVb is a level above 10% of the LDL particles.

3. The method of claim 1 wherein the LDL subclass are determined by linear gel electrophoresis, gradient gel electrophoresis, segmented gradient gel electrophoresis, NMR, analytic ultracentrifuge, or ion mobility analysis.

4. The method of claim 1 wherein the LDL IVb and LDL subclass are measured by segmented gradient gel electrophoresis.
